(19) United States
(12) Patent Application Publication
(10) Pub. No.: US 2015/0273035 A1
(43) Pub. Date: Oct. 1, 2015

(54) VACCINATION AGAINST JAPANESE ENCEPHALITIS, MEASLES, MUMPS AND RUBELLA

(71) Applicant: SANOFI PASTEUR, Lyon Cedex 07 (FR)

(72) Inventors: Emmanuel Feroldi, Ecully (FR); Alain Bouckenooghe, Singapore (SG)

(73) Assignee: SANOFI PASTEUR, Lyon (FR)

(21) Appl. No.: 14/433,256

(22) PCT Filed: Oct. 3, 2013
(86) PCT No.: PCT/EP2013/002981

§ 371 Date: Apr. 2, 2015

(30) Foreign Application Priority Data
Oct. 3, 2012 (EP) 12306204.4

(51) Int. Cl. A61K 39/00; A61K 39/25; C12N 7/00; A61K 39/12

(52) U.S. Cl. CPC A61K 39/0015; A61K 39/12; A61K 39/25; C12N 7/00; A61K 2039/5254; A61K 2039/5256; C12N 2760/18471; C12N 2760/18434; C12N 2760/18771; C12N 2760/18734; C12N 2770/36271; C12N 2770/36234; C12N 2770/24171; C12N 2770/24143; C12N 2770/24134; C12N 2710/16734

(57) ABSTRACT

The invention relates to a method of vaccinating against Japanese encephalitis (JE), measles, mumps and rubella which comprises co-administering to a patient in need, MMR vaccine and a live attenuated or inactivated cell culture derived JE vaccine.

The present invention relates to a method of vaccinating against Japanese encephalitis (JE), measles, mumps and rubella, which comprises co-administering JE, measles, mumps and rubella antigens.

Measles is a disease caused by a Paramyxovirus of the genus Morbillivirus. Measles infection runs a devastating course in children in developing countries, where the mortality rates can be as high as 2% to 15%. Pneumonia is the most common severe complication from measles and is associated with the greatest number of measles-associated deaths. The rash is intense and often hemorrhagic; it resolves after marked desquamation. Inflammation of the mucosa leads to stomatitis and diarrhea. There are other severe complications when the disease affects the brain. Measles is a vaccine preventable disease.

Mumps is a disease caused by a Paramyxovirus of the genus Rubulavirus. Among the various signs and symptoms of mumps, parotidis (inflammation of the salivary glands) which develops within 16 to 18 days after exposure to the virus is the most classical. Subjects present with fever, headache and myalgia. There might be complications such orchitis in males (more often when the virus infects adults than infants) and sterility, as well as mastitis in females. Mumps is a vaccine preventable disease.

Rubella is a disease caused by a Togavirus of the genus Rubivirus. Usually, a rash on the face and neck develops within 2 weeks after exposure to the virus. The volume of glands increases and subjects experience fever, malaise, and conjunctivitis. Rubella is thought of a benign disease, but complications including brain damages might occur in some subjects. Rubella is a vaccine preventable disease.

Measles, mumps and rubella are diseases that may be prevented by a single administration of a live attenuated measles, mumps and rubella combination vaccine, generically designated under the term MMR vaccine.

Japanese encephalitis (JE) is a disease caused by a Flavivirus. This is a mosquito-borne disease that is seasonally endemic in many countries in Southeast Asia, with three billion people living in endemic areas. Although most infections are sub-clinical, JE infection can cause a febrile illness associated with central nervous system inflammation. Only one in 250 JE infections is symptomatic in susceptible Asians; 20-30% of cases are fatal and 30-50% of survivors experience neurological or psychiatric sequelae. JE affects mainly children and teenagers, although adult cases are occasionally reported.

JE is a vaccine-preventable disease and several JE vaccines are currently in use including live attenuated and inactivated JE vaccines. The inactivated mouse brain-derived JE vaccines (MBDVs) are historically the standard-of-care JE vaccine. They comprise either the Nakayama or Beijing-1 JE virus strain grown in and purified from mouse brain and inactivated with e.g. formalin. The inactivated MBDV vaccines are produced in several countries in Asia; e.g. Thailand and South Korea (Green Cross Corporation). An MBDV Nakayama based vaccine manufactured by Biken (Osaka University, Japan) and named JE-VAX® was also licensed to and distributed by Sanofi Pasteur (Swiftwater, Pa., USA).

These MBDV vaccines are generally administered according to a prime-boost vaccination scheme including a primary immunization consisting of sequential administration of two to three doses for an adequate antibody response, followed about one or two years after the last primary dose by a booster administration to maintain long-term immunity. A typical three dose primary immunization includes a first dose at D0, a second dose at about D7, and a third dose at about D28. Abbreviated two-dose schedules have also been proposed 14 and 28 days apart.

A serological correlate of protection based on neutralising antibodies is accepted and recommended for evaluation and licensure of JE vaccines; a threshold of 1:10 using a 50% plaque reduction neutralisation test (PRNT₅₀) is accepted as evidence of protective immunity by the JE expert community. This correlate of protection has been accepted by Health Authorities for the licensure of two new vaccines (Ixiaro® from Intercell and JE-CV, IMOJEV® from Sanofi Pasteur).

In countries where measles is a life-threatening disease at a very early stage, infants are vaccinated against measles well before 12 months of age with a measles vaccine according to the national vaccination program and in their second year, they may receive an MMR for getting protection against mumps and rubella. In countries where JE is highly endemic, it is also desirable vaccinate infants against the disease.

A clinical study reported in Tseng et al, Taiwan Acta Paediatrica (1999) 40 (3): 161 has compared the immunogenicity of an inactivated mouse brain derived JE vaccine (Nakayama-based MBDV) and MMRII (Merck) when administered concomitantly or subsequently 6 weeks apart to children of about 15 months of age. Tseng et al concluded to the absence of significant differences between the two groups.

Another clinical study reported in Gatchalian et al, Vaccine (2008) 26: 2234 has shown that concomitant administration of a measles vaccine together with a live attenuated JE vaccine was feasible and effective in infants of less than 12 months of age. However, these findings cannot be extrapolated to the concomitant administration of a combination MMR vaccine and a JE vaccine, since the immunogenicity of the mumps and rubella antigens cannot be predicted from the study of Gatchalian et al. and in a different context, the immunogenicity of the measles component and that of the JE vaccine may vary. The risk of negative interference is always associated with concomitant administration of several antigens unless it is proved to the contrary. Indeed, in concomitant vaccination, even if the vaccines are administered separately, e.g., each of them being injected at different sites, there is still the potential for incompatibility among the different vaccinal agents. The immune system may be over-stimulated or inhibited and as a result, does not adequately or optimally respond to the vaccination.

Concomitant administration of MMR and JE vaccines is desirable in countries such as Singapore, Taiwan, Hong-Kong and Malaysia, wherein measles vaccination before 12 months of age is no longer prescribed and replaced by MMR vaccination at 12-18 months. The addition of a JE vaccination to the already crowed childhood vaccination schedule increases the complexity of the healthcare and raises specific issues such as compliance with the current schedule already in place, particularly in those areas of the world where regular availability of healthcare is difficult to obtain. Unfortunately, these same areas are where the threat of JE is particularly acute. Consequently, there is a desire to concomitantly administer the improved JE vaccines (not mouse brain derived) and the MMR vaccine to enhance compliance with the recommended vaccination schedule.

It has now been shown that a live attenuated measles-mumps-rubella (MMR) vaccine and a Japanese encephalitis (JE) vaccine comprising either a live attenuated JE virus or a JE virus grown on cell culture and inactivated may be safely administered in a concomitant manner at about one year of life and that the immunogenicity of each of the measles, mumps, rubella and JE viruses is not inferior to that observed for each of the four viruses when both vaccines are administered sequentially.

This is the reason why the invention relates to:
1) An MMR vaccine for use in a method of inducing a protective immune response against Japanese encephalitis which comprises co-administering to a patient in need, the MMR vaccine and a JE vaccine which is either a live attenuated JE vaccine or an inactivated, cell culture-derived JE vaccine.
2) A JE vaccine which is either a live attenuated JE vaccine or an inactivated, cell culture-derived JE vaccine, for use in a method of inducing a protective immune response against measles, mumps and rubella which comprises co-administering the JE vaccine and an MMR vaccine to a patient in need.
3) An MMR vaccine for use in a method of inducing a protective immune response against measles, mumps and rubella which comprises co-administering to a patient in need, the MMR vaccine and a JE vaccine which is either a live attenuated JE vaccine or an inactivated, cell culture-derived JE vaccine.
4) A JE vaccine which is either a live attenuated JE vaccine or an inactivated, cell culture-derived JE vaccine, for use in a method of inducing a protective immune response against Japanese encephalitis which comprises co-administering the JE vaccine and an MMR vaccine to a patient in need.
5) An MMR vaccine, for use in a method of inducing a protective immune response against measles, mumps, rubella and Japanese encephalitis which comprises co-administering to a patient in need, the MMR vaccine and a JE vaccine which is either a live attenuated JE vaccine or an inactivated, cell culture-derived JE vaccine.
6) A JE vaccine which is either a live attenuated JE vaccine or an inactivated, cell culture-derived JE vaccine, for use in a method of inducing a protective immune response against measles, mumps, rubella and Japanese encephalitis which comprises co-administering the JE vaccine and an MMR vaccine to a patient in need.

In a similar manner, the invention relates to:
7) The use of a live attenuated measles virus, a live attenuated mumps virus and a live attenuated rubella virus for use in the manufacture of an MMR vaccine for protecting an individual against measles, mumps and rubella, said MMR vaccine being intended for use in a method of inducing a protective immune response against Japanese encephalitis which comprises co-administering to a patient in need, the MMR vaccine and a JE vaccine which is either a live attenuated JE vaccine or an inactivated, cell culture-derived JE vaccine.
8) The use of a live attenuated measles virus, a live attenuated mumps virus and a live attenuated rubella virus for use in the manufacture of an MMR vaccine for protecting an individual against measles, mumps and rubella, said MMR vaccine being intended for co-administration to a patient in need, with a JE vaccine which is either a live attenuated JE vaccine or an inactivated, cell culture-derived JE vaccine.
9) The use of either a live attenuated Japanese encephalitis virus or a Japanese encephalitis virus grown on cell culture and inactivated, for use in the manufacture of a JE vaccine for protecting an individual against Japanese encephalitis; for use in a method of inducing a protective immune response against measles, mumps and rubella, which comprises co-administering the JE vaccine and an MMR vaccine to a patient in need.
10) The use of either a live attenuated Japanese encephalitis virus or a Japanese encephalitis virus grown on cell culture and inactivated, for use in the manufacture of a JE vaccine for protecting an individual against Japanese encephalitis; said JE vaccine being intended for co-administration with an MMR vaccine to a patient in need.
11) A method of inducing a protective immune response against measles, mumps, rubella and Japanese encephalitis which comprises co-administering to a patient in need:
an MMR vaccine i.a., comprising a prophylactically effective amount of a live attenuated measles virus, a live attenuated mumps virus and a live attenuated rubella virus; and
a JE vaccine i.a., comprising a prophylactically effective amount of either a live attenuated JE virus or a JE virus grown on cell culture and inactivated.
12) A method of inducing a protective immune response against measles, mumps and rubella which comprises co-administering to a patient in need:
an MMR vaccine i.a., comprising a prophylactically effective amount of a live attenuated measles virus, a live attenuated mumps virus and a live attenuated rubella virus; and
a Japanese encephalitis vaccine i.a., comprising a prophylactically effective amount of either a live attenuated JE virus or a JE virus grown on cell culture and inactivated. And
13) A method of inducing a protective immune response against Japanese encephalitis which comprises co-administering to a patient in need:
a Japanese encephalitis vaccine i.a., comprising a prophylactically effective amount of a either live attenuated JE virus or a JE virus grown on cell culture and inactivated; and
an MMR vaccine i.a., comprising a prophylactically effective amount of a live attenuated measles virus, a live attenuated mumps virus and a live attenuated rubella virus.

In another embodiment, the invention also relates to:
a) A kit comprising an MMR vaccine together with instructions for co-administering to a patient in need, the MMR vaccine and a JE vaccine which is either a live attenuated JE vaccine or an inactivated, cell culture-derived JE vaccine.
b) A kit comprising a JE vaccine which is either a live attenuated JE vaccine or an inactivated, cell culture-derived JE vaccine, optionally together with instructions for co-administering the JE vaccine and an MMR vaccine to a patient in need. And
c) A kit comprising (i) an MMR vaccine, (ii) a JE vaccine which is either a live attenuated JE vaccine or an inactivated, cell culture-derived JE vaccine, together with instructions for co-administering the MMR vaccine and the JE vaccine.

For clarity' sake, it is stated that the terms ‘co-administration’ and ‘co-administered’ are respectively equivalent to ‘concomitant administration’ and ‘concomitantly administered’.

By “co-administration” is meant administration of MMR and JE vaccines to a patient within an interval of time which may extend until at most some days (e.g., one to 6 days), preferably until at most one day, more preferably at most some hours within a day, most preferably until at most some minutes. Conveniently, co-administration is achieved during the same visit at the physician or the nurse's. Co-administration is preferably achieved at different/separate anatomic sites which are preferably drained by different lymph nodes. In practice, the MMR and JE vaccines may advantageously be administered in the upper arm and in the thigh, respectively. However, they may also be respectively administered in the left and right upper arms or in the left and right thighs or vice versa.

In each of the embodiments of the invention, the vaccines in use are conveniently co-administered as a dose. The dose is the amount of vaccinal virus which is required for inducing an immune response, i.a., a protective immune response.

An MMR vaccine may be advantageously co-administered with a JE vaccine to a patient in need from 12 to 36 months of age, preferably from 12 to 24 months of age, more preferably from 12 to 14, 15 or 18 months of age.

A live attenuated JE vaccine is a JE vaccine comprising a live attenuated JE virus. For use in the present invention, a live attenuated JE virus is advantageously grown on cell culture such as Vero or Primary Hamster Kidney (PHK) cell culture. Any of the available registered live attenuated JE vaccines may be used in the present invention.

Until recently, the only live attenuated JE vaccines currently licensed in Asian countries use the attenuated SA14-14-2 virus strain which is derived from the virulent SA14 virus strain through attenuation serial cell culture passages. Theses vaccines are generically called SA14-14-2 vaccines. An example of theses vaccines is the CD.JEVAX® manufactured by Chengdu Institute of Biological Products, People's Republic of China. The SA14-14-2 virus strain is grown in Primary Hamster Kidney (PHK) cells.

Live attenuated SA14-14-2 vaccines are usually administered in a two-dose regimen, the second dose being considered as a booster dose given from 3 to 12 months after the primary dose. The concomitant administration of a SA14-14-2 vaccine with a monovalent Measles vaccine has been achieved in infants aged 9 months in the Philippines (Gatchalian et al).

For use in the present invention, a live attenuated JE vaccine can be a chimeric vaccine. A typical example of a chimeric JE vaccine is a vaccine comprising a chimeric virus which is a live attenuated non-JE flavivirus (recipient flavivirus which is not a JE virus), in which the genetic backbone has been modified by replacing the sequences encoding the prM and/or E proteins (native prM and/or E proteins) by the sequences encoding the prM and/or E proteins of a JE virus, preferably a live attenuated JE virus, more preferably the SA14-14-2 JE virus strain.

The non-JE recipient flavivirus can be a live attenuated yellow fever virus as described in WO 98/37911 or Guirakhoo et al, Virology (1999) 257: 363. Typically, an attenuated YF virus for use in constructing a chimeric YF/JE virus may be any of the attenuated virus strains derived from the virulent YF Asibi strain and generically designated as YF-17D (Monath et al, Expert Rev. Vaccines (2005) 4: 553). Examples of useful YF viruses include the attenuated YF 17D virus (Theiler & Smith, J. Exp. Med. (1937) 65: 767-786). Examples of YF17D strains which may be used, include the YF17D204 strains (Rice et al., 1985, Science, 229: 726-733) used for example in the licensed vaccines commercialized under the trade names YF-VAX® (Sanofi-Pasteur, Swiftwater, Pa., USA), Stamaril® (Sanofi-Pasteur, Marcy I'Etoile, France), Arilvax™, (Chiron, Speke, Liverpool, UK), Flavimun® (Berna Biotech, Bern, Switzerland) and the related strains YF17DD (Genbank access number U17066), YF17D-213 (Genbank access number U17067) and strains YF17DD described by Galler et al. Vaccine (1998) 16 (9/10): 1024).

When the recipient flavivirus is a yellow fever (YF) virus, the chimeric virus is referred to as YF/JE chimera. For use in the present invention, a preferred chimeric JE virus is a live attenuated chimeric YF/JE virus which comprises the genomic backbone of an attenuated YF virus in which the nucleic acid sequences encoding the pre-membrane (prM) and envelope (E) proteins have been replaced by nucleic acid sequences encoding the corresponding structural proteins of a JE virus. Chimeras of that type involving the genetic backbone of virus strain YF 17D-204 and the prM and E proteins of strain SA14-14-2 are described for example, in patent application WO 98/37911, Chambers et al. J. of Virol. (1999) 73 (4): 3095, Monath et al, Vaccine (1999) 17: 1869, Monath et al, Vaccine (2002) 20: 1004 and Guy et al, Vaccine (2010) 28: 632. A chimeric JE virus may be conveniently grown on Vero cells. Primary immunization involving a chimeric JE vaccine may be conveniently achieved with a single dose administration.

An inactivated, cell culture-derived JE vaccine is a JE vaccine comprising a JE virus grown on cell culture and inactivated. Such vaccines are more recent than inactivated MBDVs. They include vaccines comprising as virus strain, the Nakayama, Beijing-1 or SA14-14-2 JE virus strain grown on cell culture. To this end, while any cell line appropriate for viral culture may be of use, an advantageous cell line may be the Vero cell line. Typically, viruses grown on cell culture are collected, purified and inactivated. While they may be inactivated by various means, chemical inactivation e.g. with formaldehyde, is the most common procedure. Examples of inactivated cell-culture-derived JE vaccines include Ixiaro® (Intercell, Vienna, Austria—Srivastava et al, Vaccine (2001) 19: 4557), Jebik V® (Biken, Japan—Kikukawa et al, Vaccine (2012) 30 (13): 2329) and Encevac™ (Kakesuken, Japan). Such vaccines are advantageously administered in one or two doses (primary doses), preferably two doses at least 28 days apart, the first or second primary vaccine dose being indifferently co-administered with the MMR vaccine.

MMR vaccines comprise a live attenuated measles virus, a live attenuated mumps virus and a live attenuated rubella virus. Examples of useful measles virus strains include the attenuated Enders-Edmonston, Edmonston-Zagreb and Schwarz strains and any attenuated strain derived therefrom. Examples of useful mumps virus strains include the attenuated Jeryl Lynn, Urabe AM 9, and Rubini strains and any attenuated strain derived therefrom, such as the RIT 4385 strain which is derived from the Jeryl Lynn strain. Examples of rubella virus strains include the Wistar RA 27/3 and Wistar RA 27/3M strains. In addition to the attenuated measles, mumps and rubella strains, the MMR vaccine may also comprise an attenuated varicella-zoster strain such as the Oka/Merck or Oka strain. In that case, the MMR vaccine may be designated under the term “MMRV vaccine”.

Examples of commercially available MMR vaccines include the M-M-R® II vaccine (Merck & Co, Whitehouse Station, N.J. USA), the Triviraten Berna® vaccine (also referred to as the Berna-MMR, Berna Biotech, Basel, Switzerland), the Priorix™ vaccine (Glaxo SmithKline Biologics, Rixensart, Belgium), and the Trimovax® vaccine (Sanofi Pasteur SA, Lyon, France).

Examples of commercially available MMR vaccines further comprising a varicella virus, include the ProQuad™ (Merck & Co, Whitehouse Station, N.J. USA) and the Priorix-tetra™ vaccine (Glaxo SmithKline Biologics, Rixensart, Belgium).

A long-lasting protection against measles, mumps, rubella and/or varicella is usually achieved upon administration of a single dose of a MMR vaccine to patients of 12 months of age or older.

For use in the present invention, the MMR and JE vaccines may be provided in single dose or multi-dose formulations, this later being more particularly useful for mass vaccination campaign, being understood that a dose of an MMR vaccine and a dose of a JE vaccine are concomitantly administered to a patient. When JE vaccination may be achieved according to a prime-boost scheme, the MMR vaccine is preferably co-administered with a primary JE vaccine dose.

As used herein, the terms “MMR vaccine” and “a dose of an “MMR vaccine” are used interchangeably. The terms “JE vaccine” and “a dose of a JE vaccine” are also used interchangeably, unless specified otherwise. By “dose of vaccine” is meant the amount of vaccine, that is, the amount of virus antigen that is necessary to induce an immune response. MMR and JE vaccines are advantageously lyophilized and extemporaneously reconstituted with a pharmaceutically-acceptable diluent under a volume of from 200 μl to 1.5 ml, preferably of from 0.5 to 1 ml.

### EXPERIMENTAL

1. Method

Trial Design and Participants

A phase III, randomized, multicenter open-label trial was conducted in Taiwan. In this study, one dose of JE vaccine and one dose of MMR vaccine were administered together or separately with a 6-week interval in 540 toddlers aged 12 to 18 months, with a 12-month safety and immunogenicity follow-up.

The study was aimed at demonstrating that the concomitant administration of JE and MMR vaccine has no impact on the immunogenicity of the two vaccines. In addition, the study was also aimed at assessing the potential impact of the order of administration of JE and MMR vaccine on the immunogenicity of the two vaccines.

The primary objective of the study was to demonstrate the non-inferiority of the concomitant administration of JE and MMR vaccines (Group 3 including 221 toddlers) compared to the single administration of JE and MMR (given at the first vaccination) (respectively, Group 1 including 109 toddlers and Group 2 including 217 toddlers), based on the percentage of seroconversion in a JE virus plaque reduction neutralization test (JE virus PRNT50) and on the percentage of seroconversion against measles, mumps, and rubella measured by enzyme-linked immunosorbent assays (ELISAs). Non-inferiority is demonstrated if the lower limit of the 2-sided 95% CI of the difference of seroconversion rates between groups is >−10.0%.

For JE antibody response, seroconversion was assessed 42 days after the administration of one dose of JE vaccine (D42, Group 1), and 42 days after the co-administration of JE and MMR vaccine (D42, Group 3).

Seroconversion is defined as a JE PRNT50 neutralizing antibody titer (≧10 l/dilution) in subjects who are seronegative (<10 l/dil.) at baseline. Subjects seropositive (≧10 l/dil.) at baseline require a≧four-fold rise in neutralizing antibody titers.

For measles, mumps and rubella antibody response, seroconversion was assessed 42 days after the administration of one dose of MMR vaccine (D42, Group 2), and 42 days after the co-administration of JE-CV and MMR vaccine (D42, Group 3).

Seroconversion is defined for measles, mumps and rubella respectively in subjects seronegative at baseline as an antibody titer measured by ELISA reaching the following thresholds for seropositivity at D42: ≧120 mIU/mL for measles; ≧10 ELISA units/mL for mumps; ≧10 IU/mL for rubella.

Accordingly, the defined timepoints for the immune response assessments are at baseline, i.e. before vaccine administration on Day (D) 0, then 42 days post-vaccine administration, which corresponds to D42 and D84 for Groups 1 and 2 and D42 for Group 3. In addition, the immune response is assessed 6 months after the last vaccination.

Other immunogenicity parameters (geometric mean titers [GMTs], seroprotection and seroconversion rates in JE-CV virus PRNT50 at any timepoints, the GMTs, seropositivity and seroconversion rates in MMR ELISA at any timepoints), and safety data are assessed as secondary objectives.

The persistence of neutralizing antibody titers against JE virus, measles, mumps, and rubella is assessed 6 months after the last vaccination as an observational objective.

Vaccines

IMOJEV® (JE-CV) is a live attenuated chimeric virus vaccine against Japanese Encephalitis. This chimeric virus vaccine is made of a yellow fever (YF 17D) genomic backbone in which the prM-E encoding region has been deleted and replaced by the prM-E cassette of the attenuated SA14-14-2 strain of the JE virus. The SA14-14-2 strain, including the genome sequence, has been described and used for quite a long time (Eckels et al, Vaccine (1988) 6: 513; Ni et al, J. Gen. Virol. (1995) 76: 401).

This chimera was originally constructed by Chambers et al, J. Virol. (1999) 73: 3095. The M protein exhibits a further mutation R60C, as a result of SF-Vero adaptation at P5. This R60C mutation has beneficial effect both in term of increased replication rate and improved genetic stability. The YF 17D and JE sequence junctions are at the C/prM and E/NS1 signalase cleavage sites.

IMOJEV® (JE-CV) was manufactured by Sanofi Pasteur at GPO-MBP, Thailand and supplied lyophilized as a sterile powder for injection containing a purified live attenuated chimeric YF/JE virus in stabilizing buffer containing sugars, amino acids and human serum albumin (HSA). Saline (0.4% sodium chloride solution) is used to reconstitute the vaccine. Single dose contains between 4.0 to 5.8 log₁₀ plaque forming units (PFU) of virus under a volume of 0.5 mL saline after reconstitution. A volume of 0.5 mL of the reconstituted JE-CV is administered via the subcutaneous route into the thigh.

The MMR vaccine used in the present study is MMRII®, manufactured by Merck & Co. It is supplied as a sterile lyophilized preparation of i) Attenuvax® (live attenuated measles virus), a more attenuated line of measles virus, derived from Enders' attenuated Edmonston strain and propagated in chick embryo cell culture; ii) Mumpsvax® (live attenuated mumps virus), the Jeryl Lynn™ (B level) strain of mumps virus propagated in chick embryo cell culture; and iii) Meruvax® II (live attenuated rubella virus), the Wistar RA 27/3 strain of live attenuated rubella virus propagated in WI-38 human diploid lung fibroblasts. MMRII® is licensed in Taiwan and included in the national immunization schedule.

Each dose of the vaccine contains sorbitol (14.5 mg), sodium phosphate, sucrose (1.9 mg), sodium chloride, hydrolyzed gelatine (14.5 mg), recombinant human albumin (0.3 mg), foetal bovine serum (<1 parts per million [ppm]), other buffer and media ingredients and approximately 25 μg of neomycin. The product contains no preservative.

Lyophilized MMR vaccine is reconstituted according to the manufacturer's instructions as a 0.5 mL dose prior to injection. Each 0.5 mL dose of reconstituted vaccine contains i) at least 1000 cell culture infectious dose 50% (CCID₅₀) measles virus; ii) at least 20,000 CCID₅₀ mumps virus; and at least 1000 CCID₅₀ rubella virus. The reconstituted MMR vaccine is administered via the subcutaneous route into the upper arm (deltoid).

Immunogenicity Assessment Methods

For JE-CV Antibody Response: PRNT50 Using the JE-CV Virus

JE virus neutralizing antibody measurement was assessed by JE neutralizing antibody PRNT50 test by Focus Diagnostics Inc., Cypress, Calif., USA using the homologous virus (JE-CV).

Serial, 2-fold dilutions of serum to be tested (previously heat-inactivated) are mixed with a constant challenge dose of a JE-CV virus (expressed as PFU/mL). The mixtures are inoculated into wells of a 24-well plate of confluent Vero cells. After adsorption, cell monolayers are overlaid, incubated for 5 days, and then stained with crystal violet/formaldehyde solution. The neutralizing antibody titer is calculated and expressed as the reciprocal serum dilution reducing the mean plaque count by 50% as compared to the mean virus plaque number with a JE antibody negative control serum. The lower limit of quantitation (LLOQ) for this study is 10 l/dil.

For Measles, Mumps and Rubella Antibody Response: MMR ELISAs

MMR antibody measurements were performed at Pharmaceutical Product Development (PPD), Wayne, Pa., USA.

Measles, mumps and rubella antibodies in serum samples were quantified using ELISA assays. These assays follow the same principle with the coating antigen dependent upon the assay: either measles virus, mumps virus or rubella virus. Inactivated viral antigen is adsorbed to wells of a solid phase microtiter plate. Specific antibodies in the reference standard, serum quality controls and test samples bind to the immobilized antigen, unbound antibodies are washed from the wells, and enzyme-conjugate anti-human immunoglobulin (Ig) is added. The enzyme conjugate binds to the antigen-antibody complex. Excess conjugate is washed away and a specific colorimetric substrate is added. Bound enzyme catalyzes a hydrolytic reaction that causes colour development. After a specific time, the reaction is stopped. The intensity of the colour is proportional to the amount of specific antibody bound to the wells. The results are read on a spectrophotometer (ELISA plate reader). For each test sample, or control sample, quantification of the human IgG antibody to virus or titer is determined by comparison of the resulting test optical density to a standard curve generated using the reference serum.

2. Results

42 days after co-administration, 96.9% (95% CI: 93.4; 98.9) of subjects seroconverted to JE as did 97.9% (92.6; 99.7) of those receiving JE-CV alone. In addition, 42 days after co-administration, there were 100.0% (98.1; 100.0), 99.5% (97.2; 100.0) and 99.4% (96.9; 100.0) seroconversion against measles, mumps and rubella, respectively; in subjects receiving MMR alone, seroconversion rates were 97.6 (94.1; 99.4), 98.8 (95.9; 99.9), and 99.4 (96.4; 100.0), respectively. Non-inferiority of immune responses in terms of seroprotection rates is therefore demonstrated for all antigens. JE-CV GMT were slightly lower in subjects who received concomitantly JE-CV and MMR as regards to other groups, but without clinical significance as values were far above the seroprotective threshold. GMTs were in similar ranges for Measles, Mumps and Rubella, irrespective of a concomitant administration or not.

Seroconversion rates (% of subjects who seroconverted), seroprotection rates are provided and GMTs are provided hereinafter in Tables 1, 2 and 3.

As shown in Table 1, non-inferiority of the concomitant administration of JE and MMR vaccines (Group 3) compared to the single administration of JE and MMR is demonstrated since the lower limit of the 2-sided 95% CI of the difference between the groups is >−10.0%, for each antigen (JE, measles, mumps and rubella).

TABLE 1

| | | | Sequential | Sequential | JConcomitant | |
|---|---|---|---|---|---|---|
| | | | JE-CV/MMR | MMR/JE-CV | JE-CV + MMR | |
| | | | Group 1 | Group 2 | Group 3 | |
| % | N | Criteria | 102 | 217 | 221 | |
| JE-CV | D0 | <10 (1/dil) | 100 [96.2; 100.0] | | 100.0 | [98.0; 100.0] |
| PRNT50 | D42 | Seroconversion | 97.9 [92.6; 99.7] | | 96.9 | [93.4; 98.9] |
| | | Gp3-Gp1: | | | −1.0 | [−4.8; 4.5] |
| Measles | D0 | <120 mLU/mL | | 97.2 [93.5; 99.1] | 99. | [0 96.4; 99.9] |
| ELISA | D42 | Seroconversion | | 97.6 [94.1; 99.4] | 100.0 | [98.1; 100.0] |
| | | Gp3-Gp2: | | | 2.4 | [−0.1; 5.9] |
| Mumps | D0 | <10 units/mL | | 98.9 [96.0; 99.9] | 99.5 | [97.2; 100.0] |
| ELISA | D42 | Seroconversion | | 98.8 [95.9; 99.9] | 99.5 | [97.2; 100.0] |
| | | Gp3-Gp2: | | | 0.6 | [−1.9; 3.6] |
| Rubella | D0 | <10 IU/mL | | 88.1 [82.3; 92.5] | 91.3 | [86.5; 94.9] |
| ELISA | D42 | Seroconversion | | 99.4 [96.4; 100.0] | 99.4 | [96.9; 100.0] |
| | | Gp3-Gp2: | | | 0.1 | [−2.5; 3.1] |

TABLE 2
Seroprotection up to six months

| | | Sequential | | Sequential | Concomitant | |
|---|---|---|---|---|---|---|
| | | JE-CV/MMR | | MMR/JE-CV | JE-CV + MMR | |
| | | Group 1 | | Group 2 | Group 3 | |
| % | N | 102 | | 217 | 221 | |
| JE-CV | D0 | 2.00 | [0.2; 6.9] | | 1.8 | [0.5; 4.6] |
| PRNT50 | D42 | 98.0 | [93.0; 99.8] | 5.1 [2.5; 9.1] | 95.5 | [91.8; 97.8] |
| | D84 | | | 100.0 [96.4; 100.0] | | |
| | M6 | 95.8 | [89.7; 98.9] | 99.5 [97.1; 100.0] | 93.5 | [89.4; 96.4] |
| Measles ELISA | D0 | | | 3.7 1.6; 7.1 | | |
| | D42 | 4.0 | [1.1; 9.8] | 98.0 [94.9; 99.4] | 100.0 | [98.3; 100.0] |
| | D84 | 98.0 | [93.0; 99.8] | | | |
| | M6 | 96.9 | [91.1; 99.4] | 96.0 [96.3; 99.9] | 99.5 | [97.5; 100.0] |
| Mumps ELISA | D0 | | | 0.9 [0.1; 3.3] | 0.9 | [0.1; 3.3] |
| | D42 | 1.0 | [0.0; 5.4] | 99.0 96.4; 99.9 | 99.5 | [97.5; 100.0] |
| | D84 | 100.0 | [98.1; 100.0] | | | |
| | M6 | 93.8 | [86.9; 97.7] | 94.2 [89.9; 97.1] | 95.9 | [92.3; 98.1] |
| Rubella ELISA | D0 | | | 10.6 6.8; 15.5 | 8.7 | [5.3; 13.2] |
| | D42 | 9.9 | [4.9; 17.5] | 99.5 [97.2; 100.0] | 99.5 | [97.5; 100.0] |
| | D84 | 100.0 | [96.4; 100.0] | | | |
| | M6 | 99.0 | [94.3; 100.0] | 100.0 [98.1; 100.0] | 100.0 | [98.3; 100.0] |

As shown in Table 2, seroprotection rates observed after concomitant administration (Group 3) remain as high as those observed after sequential administration (Groups 1 and 2), up to at least six months.

TABLE 3
GMTs up to six months

| | | Sequential | | Sequential | | Concomitant | |
|---|---|---|---|---|---|---|---|
| | | JE-CV/MMR | | MMR/JE-CV | | JE-CV + MMR | |
| | | Group 1 | | Group 2 | | Group 3 | |
| % | N | 102 | | 217 | | 221 | |
| JE-CV | D0 | 5.21 | [4.90; 5.53] | | | 5.16 | [4.99; 5.33] |
| PRNT50 | D42 | 510 | [361; 721] | 5.80 | [5.23; 6.42] | 304 | [239; 387] |
| | D84 | | | 579 | [458; 732] | | |
| | M6 | 298 | [212; 418] | 343 | [277; 424] | 203 | [161; 255] |
| Measles ELISA | D0 | | | 34.2 | [32.2; 36.2] | 33.5 | [32.1; 34.9] |
| | D42 | 34.5 | [31.7; 37.4] | 1727 | [1526; 1955] | 1953 | [1790; 2131] |
| | D84 | 1878 | [1585; 2225] | | | | |
| | M6 | 1165 | [955; 1421] | 1116 | [987; 1262] | 1269 | [1145; 1405] |
| Mumps ELISA | D0 | | | 2.62 | [2.52; 2.71] | 2.56 | [2.50; 2.63] |
| | D42 | 2.58 | [2.47; 2.70] | 90.9 | [81.3; 102] | 94.5 | [85.1; 105] |
| | D84 | 78.3 | [67.9; 90.3] | | | | |
| | M6 | 46.2 | [38.6; 55.3] | 51.0 | [44.4; 58.7] | 47.1 | [41.5; 53.3] |
| Rubella ELISA | D0 | | | 3.88 | [3.57; 4.22] | 3.61 | [3.35; 3.90] |
| | D42 | 3.87 | [3.43; 4.38] | 83.3 | [75.9; 91.4] | 69.9 | [64.3; 76.0] |
| | D84 | 78.9 | [69.6; 89.4] | | | | |
| | M6 | 99.5 | [87.5; 113] | 109 | [99.7; 119] | 95.2 | [88.9; 102] |

As shown in Table 3, JV-CV GMTs are slightly lower in subjects who received concomitantly JE-CV and MMR as regards to other groups. However, the difference has no clinical relevance when considering the six month-follow-up. No interference is observed in the immune responses up to six month-follow-up when vaccines are given concomitantly or sequentially.

### CONCLUSION

The concomitant administration of JE-CV (IMOJEV®) with MMR®II vaccines in 12-18 month-olds shows no differences in seroconversion rates in comparison to sequential administrations. Moreover, the study shows there is no impact due to the order of vaccine administration. The co-administration of JE-CV and MMR vaccines could be recommended to facilitate immunization of children against these diseases at a single visit.

1. A live attenuated measles-mumps-rubella (MMR) vaccine for use in a method of inducing a protective immune response against Japanese encephalitis, said method comprising co-administering to a patient in need the MMR vaccine and a Japanese encephalitis (JE) vaccine which is either a live attenuated JE vaccine or an inactivated cell culture derived JE vaccine.

2. An MMR vaccine according to claim 1, wherein the patient is from 12 to 36 months of age.

3. An MMR vaccine according to claim 2, wherein the patient is from 12 to 18 months of age.

4. An MMR vaccine according to claim 1, wherein the JE vaccine is an inactivated cell culture derived JE vaccine.

5. An MMR vaccine according to claim 4, wherein the JE vaccine is an inactivated Vero cell culture derived JE vaccine.

6. An MMR vaccine according to claim 4, wherein the JE vaccine comprises the SA14-14-2 JE virus strain grown on cell culture and inactivated.

7. An MMR vaccine according to claim 1, wherein the JE vaccine is a live attenuated JE vaccine.

8. An MMR vaccine according to claim 7, wherein the live attenuated JE vaccine comprises the live attenuated SA 14-14-2 JE virus strain.

9. An MMR vaccine according to claim 7, wherein the live attenuated JE vaccine is a chimeric vaccine.

10. An MMR vaccine according to claim 9, wherein the live attenuated chimeric JE vaccine comprises a chimeric virus which is a live attenuated non-JE flavivirus in which the genetic backbone has been modified by replacing the sequences encoding the prM and/or E proteins with the sequences encoding the prM and/or E proteins of a JE virus.

11. An MMR vaccine according to claim 10, wherein the JE virus is attenuated.

12. An MMR vaccine according to claim 11, wherein the attenuated JE virus is the SAM-14-2 virus strain.

13. An MMR vaccine according to claim 5, wherein the live attenuated non-JE flavivirus is a live attenuated yellow fever virus.

14. An MMR vaccine according to claim 13, wherein the live attenuated yellow fever Virus is an YF-17D virus.

15. An MMR vaccine according to claim 1, wherein the method comprises co-administering a primary dose of the JE vaccine and a dose of the MMR vaccine.

16. A Japanese encephalitis (JE) vaccine which is either a live attenuated JE vaccine or an inactivated cell culture derived JE vaccine, for use in a method of inducing a protective immune response against measles, mumps and rubella, said method comprising co-administering the Japanese encephalitis vaccine and an MMR vaccine to a patient in need.

17. A JE vaccine according to claim 16, wherein the patient is from 12 to 36 months of age.

18. A JE vaccine according to claim 17, wherein the patient is from 12 to 18 months of age.

19. A JE vaccine according to claim 16, which is an inactivated cell culture derived JE vaccine.

20. A JE vaccine according to claim 19, which is an inactivated Vero cell culture derived JE vaccine.

21. A JE vaccine according to claim 19, which comprises the SA14-14-2 JE virus strain grown on cell culture and inactivated.

22. A JE vaccine according to claim 16, which is a live attenuated JE vaccine.

23. A JE vaccine according to claim 22, which comprises the live attenuated SA14-14-2 JE virus strain.

24. A JE vaccine according to claim 22, which is a chimeric vaccine.

25. A JE vaccine according to claim 24, which comprises a chimeric virus which is a live attenuated non-JE flavivirus in which the genetic backbone has been modified by replacing the sequences encoding the prM and/or E proteins with the sequences encoding the prM and/or E proteins of a JE virus.

26. A JE vaccine according to claim 25, wherein the JE virus is attenuated.

27. A JE vaccine according to claim 26, wherein the attenuated JE virus is the SAM-14-2 virus strain.

28. A JE vaccine according to claim 25, wherein the live attenuated non-JE flavivirus is a live attenuated yellow fever virus.

29. A JE vaccine according to claim 28, wherein the live attenuated yellow fever Virus is a YF-17D virus.

30. A JE vaccine according to claim 16, wherein the method comprises co-administering a primary dose of the JE vaccine and a dose of the MMR vaccine.

31. A JE vaccine according to claim 16, wherein the MMR vaccine comprises a live attenuated varicella zoster virus.

32. An MMR vaccine according to claim 1, which further comprises a live attenuated varicella zoster virus.
